(19) United States
(12) Patent Application Publication
(10) Pub. No.: US 2026/0092328 A1
(43) Pub. Date: Apr. 2, 2026

(54) MICRORNA BIOMARKERS FOR MULTI CANCER EARLY DETECTION TEST (MCED)

(71) Applicant: UNIVERSITY OF SOUTHAMPTON, Hampshire (GB)

(72) Inventors: Andrew George SHAPANIS, Hampshire (GB); Paul James Stuart SKIPP, Hampshire (GB)

(21) Appl. No.: 19/123,700

(22) PCT Filed: Oct. 24, 2023
(86) PCT No.: PCT/GB2023/052783

§ 371 Date: Apr. 23, 2025

(30) Foreign Application Priority Data
Oct. 24, 2022 (GB) 2215749.9

(51) Int. Cl. C12Q 1/6886

(52) U.S. Cl. CPC C12Q 1/6886; C12Q 2600/158; C12Q 2600/178

(57) ABSTRACT

The invention relates to a method of diagnosing a cancer in a subject, wherein the method comprises determining the expression level of a set of miRNAs in a sample obtained from the subject. The invention also relates to a method of identifying the site of localisation of a cancer in a subject. The invention also provides method of treating a subject with cancer.

### TECHNICAL FIELD

The present invention relates to novel biomarkers and their use in diagnosing cancer in a subject.

### INTRODUCTION

The diagnosis of cancer currently relies heavily on screening methods, which generally only look for specific cancers; this is the case for breast, bowel and cervical cancer which are screened for periodically in the UK and the USA. As well as being applicable for a certain type of cancer which is suspected, results must be followed up with more expensive diagnostic tests and also suffer poor specificity, resulting in unnecessary costs, as well as stress for the patient.

Only very limited technologies are available for use as a multi cancer early diagnostic (MCED). One such option is a serum based diagnostic test, the Galleri test (GRAIL) which is only available in certain territories and is expensive (approx. USD 950 per test). Additionally, sensitivity for detection of stage one cancer is poor (18-39%).

Other tests in development include CancerSEEK (ThriveDetect) and PanSeer (Singlera). The Galleri and PanSeer tests are based on circulating methylated DNA to diagnose cancer and CancerSEEK uses proteins and mutation in circulating DNA. Investigating methylation states of circulating DNA is costly and requires extensive user experience. The CancerSEEK system is also expensive to purchase, run and maintain.

Early detection of cancer is one of the greatest unmet needs in today's cancer care regime. Diagnosing cancer before it becomes too aggressive would not only save countless lives but would also negate the costly and traumatic process of late stage and palliative cancer care.

There is therefore a need to provide new methods of detecting cancer in patients, which are highly sensitive and accurate, and which are relatively low-cost.

### SUMMARY OF INVENTION

In an aspect, the invention provides a method of diagnosing a cancer in a subject, wherein the method comprises:
i. providing a biological sample obtained from the subject;
ii. determining the expression level of at least three miRNAs in the sample obtained from the subject;
iii. using the results from (ii) to determine if the subject has a cancer.

In another aspect, there is provided a method of treating a subject with cancer, wherein the method comprises:
i. providing a biological sample obtained from the subject;
ii. determining the expression level of at least three miRNAs in the sample obtained from the subject;
iii. using the results from (ii) to determine if the subject has a cancer; and
iv. administering an anti-cancer therapeutic to the subject if the subject is determined to have cancer.

In any aspect, a machine learning model may be used to determine if the subject has cancer from the expression levels of the at least three miRNAs.

The machine learning model may comprise a support vector machine. The support vector machine may employ a radial kernel.

The machine learning model for determining if the subject has cancer may be a first machine learning model. The method may comprise using a second machine learning model to identify what type (i.e. localisation) of cancer is present in the event that the first machine learning model determines that the subject has cancer.

The second machine learning model may comprise a feedforward artificial neural network. The feedforward artificial neural network may comprise a single hidden layer, and/or at least one hidden layer.

In some embodiments, the machine learning model may be an integrated machine learning model, which both determines whether the subject has cancer and identifies what type of cancer is present.

Any of the machine learning models may be trained using training data, comprising miRNA expression levels for the at least 3 miRNAs for subjects with known cancer diagnoses. The known cancer diagnoses may comprise at least 5 different cancer types, or at least 10 cancer types. The different cancer types may comprise at least one of: bladder cancer, bone and soft tissue sarcoma, breast cancer, colorectal cancer, esophageal cancer, gastric cancer, glioblastoma, HCC, lung cancer, ovarian cancer, pancreatic cancer, and prostate cancer.

The at least three miRNAs may be selected from Table 1.

The at least three miRNAs may comprise or consist of three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 of the miRNAs in Table 1.

The at least three miRNAs may comprise or consist of three miRNAs selected from SEQ ID NOs: 1-10.

The at least three miRNAs may comprise or consist of three miRNAs selected from SEQ ID NOs: 1-9.

The at least three miRNAs may comprise or consist of three miRNAs selected from SEQ ID NOs: 1-8.

The at least three miRNAs may comprise or consist of three miRNAs selected from SEQ ID NOs: 1-7

The at least three miRNAs may comprise or consist of four miRNAs selected from SEQ ID NOs: 1-10.

The at least three miRNAs may comprise or consist of four miRNAs selected from SEQ ID NOs: 1-9.

The at least three miRNAs may comprise or consist of four miRNAs selected from SEQ ID NOs: 1-8.

The at least three miRNAs may comprise or consist of four miRNAs selected from SEQ ID NOs: 1-7

The at least three miRNAs may comprise or consist of five miRNAs selected from SEQ ID NOs: 1-10.

The at least three miRNAs may comprise or consist of five miRNAs selected from SEQ ID NOs: 1-9.

The at least three miRNAs may comprise or consist of five miRNAs selected from SEQ ID NOs: 1-8.

The at least three miRNAs may comprise or consist of five miRNAs selected from SEQ ID NOs: 1-7

The at least three miRNAs may comprise or consist of miRNAs having the sequence of SEQ ID NO: 1, SEQ ID NO: 3, SEQ ID NO: 4, SEQ ID NO: 5, and SEQ ID NO: 6.

The at least three miRNAs may comprise or consist of miRNAs having the sequence of SEQ ID NO: 1, SEQ ID NO: 2 and SEQ ID NO: 3.

The at least three miRNAs may comprise or consist of miRNAs having the sequence of SEQ ID NO: 1, SEQ ID NO: 2, SEQ ID NO: 3 and SEQ ID NO: 4.

The at least three miRNAs may comprise or consist of miRNAs having the sequence of SEQ ID NO: 1, SEQ ID NO: 2, SEQ ID NO: 3, SEQ ID NO: 4 and SEQ ID NO: 5.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, and SEQ ID NO: 6.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6 and SEQ ID NO: 7.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8.

The at least three miRNAs may comprise or consist of miRNAs having the sequence of SEQ ID NO: 1, SEQ ID NO: 2, SEQ ID NO: 3, SEQ ID NO: 4, SEQ ID NO: 5 SEQ ID NO: 6, SEQ ID NO: 7, SEQ ID NO: 8 and SEQ ID NO: 9.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 and SEQ ID NO: 10.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and SEQ ID NO: 11.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 and SEQ ID NO: 12.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 and SEQ ID NO: 13.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 and SEQ ID NO: 14.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 and SEQ ID NO: 15.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 17.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17 and SEQ ID NO: 18.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19 and SEQ ID NO: 20.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21 and SEQ ID NO: 22

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 and SEQ ID NO: 23.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23 and SEQ ID NO: 24.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24 and SEQ ID NO: 25.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and SEQ ID NO: 26.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26 and SEQ ID NO: 27.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 and SEQ ID NO: 28.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28 and SEQ ID NO: 29.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30 and SEQ ID NO: 31.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, and SEQ ID NO: 32.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 and SEQ ID NO: 33.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33 and SEQ ID NO: 34.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 and SEQ ID NO: 35.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 and SEQ ID NO: 36.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36 and SEQ ID NO: 37.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 SEQ ID NO: 36, SEQ ID NO: 37 and SEQ ID NO: 38.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 SEQ ID NO: 36, SEQ ID NO: 37, SEQ ID NO: 38 and SEQ ID NO: 39.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 SEQ ID NO: 36, SEQ ID NO: 37, SEQ ID NO: 38, SEQ ID NO: 39 and SEQ ID NO: 40.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 SEQ ID NO: 36, SEQ ID NO: 37, SEQ ID NO: 38, SEQ ID NO: 39, SEQ ID NO: 40 and SEQ ID NO: 41.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 SEQ ID NO: 36, SEQ ID NO: 37, SEQ ID NO: 38, SEQ ID NO: 39, SEQ ID NO: 40, SEQ ID NO: 41 and SEQ ID NO: 42.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 SEQ ID NO: 36, SEQ ID NO: 37, SEQ ID NO: 38, SEQ ID NO: 39, SEQ ID NO: 40, SEQ ID NO: 41, SEQ ID NO: 42 and SEQ ID NO: 43.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43 and SEQ ID NO: 44.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44 and SEQ ID NO: 45.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 SEQ ID NO: 36. SEQ ID NO: 37, SEQ ID NO: 38, SEQ ID NO: 39, SEQ ID NO: 40, SEQ ID NO: 41. SEQ ID NO: 42, SEQ ID NO: 43, SEQ ID NO: 44, SEQ ID NO: 45, SEQ ID NO: 46 and SEQ ID NO: 47.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 SEQ ID NO: 36. SEQ ID NO: 37, SEQ ID NO: 38, SEQ ID NO: 39, SEQ ID NO: 40, SEQ ID NO: 41. SEQ ID NO: 42, SEQ ID NO: 43, SEQ ID NO: 44, SEQ ID NO: 45, SEQ ID NO: 46. SEQ ID NO: 47 and SEQ ID NO: 48.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 SEQ ID NO: 36. SEQ ID NO: 37, SEQ ID NO: 38, SEQ ID NO: 39, SEQ ID NO: 40, SEQ ID NO: 41. SEQ ID NO: 42, SEQ ID NO: 43, SEQ ID NO: 44, SEQ ID NO: 45, SEQ ID NO: 46. SEQ ID NO: 47, SEQ ID NO: 48 and SEQ ID NO: 49.

The at least three miRNAs may comprise or consist of miRNAs having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49 and SEQ ID NO: 50.

In another aspect, the invention provides a method of determining the localisation of a cancer in a subject, comprising:
i. providing a biological sample obtained from the subject;
ii. determining the expression level of a panel of miRNAs, the panel comprising miRNAs having the sequences of three or more of, up to all of, SEQ ID NOs 1-50 in the sample obtained from the subject; and
iii. using the results from (ii) to determine a location of the cancer.

Step iii. of the method may comprise using a second machine learning model to identify what type (i.e. localisation) of cancer is present in the event that the first machine learning model determines that the subject has cancer.

The second machine learning model may comprise a feedforward artificial neural network. The feedforward artificial neural network may comprise a single hidden layer, and/or at least one hidden layer.

The localisation of a cancer may refer to the part of the body where a primary tumour establishes and/or develops.

In an embodiment, the panel may comprise a subset of the SEQ ID Nos 1-50. The subset may comprise or consist of all, or at least some, of the 30 miRNAs of Table 2. The subset may comprise or consist of the first 25 miRNAs of Table 2. The subset may comprise or consist of the first 20 miRNAs of Table 2. The subset may comprise or consist of the first 15 miRNAs of Table 2. The subset may comprise or consist of the first 10 miRNAs of Table 2.

In any aspect, the biological sample may be a biological fluid, such as a blood, plasma, serum, urine or cerebrospinal fluid sample. Preferably, the biological sample is a serum sample.

In any aspect, the cancer may be a solid cancer. The solid cancer be selected from the group consisting of: bladder cancer, bone and soft tissue sarcoma, breast cancer, colorectal cancer, oesophageal cancer, gastric cancer, glioblastoma, hepatocellular carcinoma (HCC), lung cancer, ovarian cancer, pancreatic cancer, or prostate cancer.

In any aspect, the cancer may be stage 0 cancer, stage 1 cancer, stage 2 cancer, stage 3 cancer or stage 4 cancer.

In any aspect, the level of one or more miRNA in a sample obtained from a subject may be determined using any suitable assay known to the skilled person. For example, the level of one or more miRNA in a sample obtained from a subject may be determined using microarray analysis, qRT-PCR, Next Generation Sequencing (NGS), NanoString, branched DNA analysis or RNASeq.

In another aspect, there is provided a kit for carrying out the method of any aspect of the invention. The kit may further comprise a set of instructions. The instructions may set out to the reader the details and information required to perform any method described herein, using the contents of the kit and/or other reagents which are available to the reader. The kit may comprise one or more reagents, such as oligonucleotide primers, for determining the expression level of miRNAs which are to be detected in the method. Each oligonucleotide primer may be specific for a miRNA of the method.

The skilled person will appreciate that preferred features of any one embodiment and/or aspect of the invention may be applied to all other embodiments and/or aspects of the invention.

TABLE 1
Panel of 50 miRNA biomarkers important for
performing the methods of the invention

| | | SEQ ID |
|---|---|---|
| Accession | miRNA Sequence | NO: |
| MIMAT0027511 | UUGCUCUGCUCCCCCGCCCCCAG | 1 |
| MIMAT0025856 | CUGGGCCCGCGGCGGGCGUGGGG | 2 |
| MIMAT0027430 | GUGAGGCGGGGCCAGGAGGGUGUGU | 3 |
| MIMAT0018987 | GAGACUGGGGUGGGGCC | 4 |
| MIMAT0019032 | CUCCGGGACGGCUGGGC | 5 |
| MIMAT0004609 | AGGGAGGGACGGGGGCUGUGC | 6 |
| MIMAT0027404 | GGGGGGUGUGGAGCCAGGGGGC | 7 |
| MIMAT0027442 | CUCGGGAGGGCAUGGGCCAGGC | 8 |
| MIMAT0027470 | UGGGAGGGCGUGGAUGAUGGUG | 9 |
| MIMAT0027590 | CGGGGCCAGAGCAGAGAGC | 10 |
| MIMAT0022259 | UUCAGAUCCCAGCGGUGCCUCU | 11 |
| MIMAT0015082 | GGAGGCGCAGGCUCGGAAAGGCG | 12 |
| MIMAT0019807 | GCCCCGGCGCGGGCGGGUUCUGG | 13 |
| MIMAT0027462 | CGGGCCGGAGGUCAAGGGCGU | 14 |
| MIMAT0019878 | UGAGUGGGGCUCCCGGGACGGCG | 15 |
| MIMAT0018076 | GGCGGGUGCGGGGGUGG | 16 |
| MIMAT0018929 | GGUGGGCUUCCCGGAGGG | 17 |
| MIMAT0019699 | UGGGCCAGGGAGCAGCUGGUGGG | 18 |
| MIMAT0018925 | CGGGCGUGGUGGUGGGGGUG | 19 |
| MIMAT0027650 | UGAGGGACCCAGGACAGGAGA | 20 |
| MIMAT0004561 | GGCUACAACACAGGACCCGGGC | 21 |
| MIMAT0018085 | UGAGCACCACACAGGCCGGGCGC | 22 |
| MIMAT0022713 | AUAUACAGGGGGAGACUCUCAU | 23 |
| MIMAT0019776 | CUCCUGGGGCCCGCACUCUCGC | 24 |
| MIMAT0007883 | CGCAGGGGCCGGGUGCUCACCG | 25 |
| MIMAT0027426 | CUGGGGAGUGGCUGGGGAG | 26 |
| MIMAT0018999 | GGUGGGGGGUGUUGUUUU | 27 |
| MIMAT0001339 | ACUGGACUUAGGGUCAGAAGGC | 28 |
| MIMAT0000278 | AGCUACAUUGUCUGCUGGGUUUC | 29 |
| MIMAT0019859 | GCUGCGGGCUGCGGUCAGGGCG | 30 |
| MIMAT0027562 | UAGGUAGAGUGUGAGGAGGAGGUC | 31 |
| MIMAT0004952 | ACCAGGAGGCUGAGGCCCCU | 32 |
| MIMAT0019815 | GGGUGAGGGCAGGUGGUU | 33 |
| MIMAT0000069 | UAGCAGCACGUAAAUAUUGGCG | 34 |
| MIMAT0023712 | UGAGGCGGGGGGGCGAGC | 35 |
| MIMAT0022838 | AUAUACAGGGGGAGACUCUUAU | 36 |
| MIMAT0027387 | AAGGGGCAGGGACGGGUGGCCC | 37 |
| MIMAT0019073 | GGAUGGAGGAGGGGUCU | 38 |
| MIMAT0015079 | CGCGCCGGGCCCGGGUU | 39 |
| MIMAT0019775 | UGGCUGUUGGAGGGGGCAGGC | 40 |
| MIMAT0027575 | AUGCCUCCCCCGGCCCCGCAG | 41 |
| MIMAT0027506 | CUGGGGGUGGGGGGCUGGGCGU | 42 |
| MIMAT0023713 | AGAGAUGAAGCGGGGGGGCG | 43 |
| MIMAT0000461 | UAGCAGCACAGAAAUAUUGGC | 44 |
| MIMAT0019849 | UGGGAGGGGAGAGGCAGCAAGCA | 45 |
| MIMAT0005951 | ACUCGGCGUGGCGUCGGUCGUG | 46 |
| MIMAT0022943 | AGUGGGAGGCCAGGGCACGGCA | 47 |
| MIMAT0022938 | GUGAGUCAGGGUGGGGCUGG | 48 |
| MIMAT0027373 | CUGGGUGAGGGCAUCUGUGGU | 49 |
| MIMAT0027436 | CACACAGGAAAAGCGGGGCCCUG | 50 |

TABLE 2
miRNAs identified as important for
determining the localisation of a cancer.

| | | SEQ ID |
|---|---|---|
| Accession | miRNA Sequence | NO: |
| MIMAT0023712 | UGAGGCGGGGGGGCGAGC | 35 |
| MIMAT0027575 | AUGCCUCCCCCGGCCCCGCAG | 41 |
| MIMAT0000278 | AGCUACAUUGUCUGCUGGGUUUC | 29 |
| MIMAT0022838 | AUAUACAGGGGGAGACUCUUAU | 36 |
| MIMAT0022713 | AUAUACAGGGGGAGACUCUCAU | 23 |
| MIMAT0000461 | UAGCAGCACAGAAAUAUUGGC | 44 |
| MIMAT0001339 | ACUGGACUUAGGGUCAGAAGGC | 28 |
| MIMAT0019815 | GGGUGAGGGCAGGUGGUU | 33 |
| MIMAT0023713 | AGAGAUGAAGCGGGGGGGCG | 43 |
| MIMAT0027436 | CACACAGGAAAAGCGGGGCCCUG | 50 |
| MIMAT0018999 | GGUGGGGGGUGUUGUUUU | 27 |
| MIMAT0019776 | CUCCUGGGGCCCGCACUCUCGC | 24 |
| MIMAT0019073 | GGAUGGAGGAGGGGUCU | 38 |
| MIMAT0000069 | UAGCAGCACGUAAAUAUUGGCG | 34 |
| MIMAT0027562 | UAGGUAGAGUGUGAGGAGGAGGUC | 31 |
| MIMAT0019878 | UGAGUGGGGCUCCCGGGACGGCG | 15 |
| MIMAT0007883 | CGCAGGGGCCGGGUGCUCACCG | 25 |
| MIMAT0022943 | AGUGGGAGGCCAGGGCACGGCA | 47 |
| MIMAT0018085 | UGAGCACCACACAGGCCGGGCGC | 22 |
| MIMAT0027470 | UGGGAGGGCGUGGAUGAUGGUG | 9 |
| MIMAT0005951 | ACUCGGCGUGGCGUCGGUCGUG | 46 |
| MIMAT0025856 | CUGGGCCCGCGGCGGGCGUGGGG | 2 |
| MIMAT0019775 | UGGCUGUUGGAGGGGGCAGGC | 40 |
| MIMAT0004561 | GGCUACAACACAGGACCCGGGC | 21 |
| MIMAT0019849 | UGGGAGGGGAGAGGCAGCAAGCA | 45 |
| MIMAT0015079 | CGCGCCGGGCCCGGGUU | 39 |
| MIMAT0022938 | GUGAGUCAGGGUGGGGCUGG | 48 |
| MIMAT0027373 | CUGGGUGAGGGCAUCUGUGGU | 49 |
| MIMAT0019859 | GCUGCGGGCUGCGGUCAGGGCG | 30 |
| MIMAT0027590 | CGGGGCCAGAGCAGAGAGC | 10 |

The invention is based on the inventors' finding that assessing the levels of expression of different miRNAs in the blood/serum of a patient can be used advantageously as a multi cancer early diagnostic (MCED). To this date, no other MCED using serum can diagnose cancer by looking at circulating miRNA levels in the blood. The inventors' test is as specific as an existing MCED, the Galleri test, but has significantly higher sensitivity than this and other tests (CancerSEEK and Elypta) at all stages (see FIG. 5).

Further still, the methods of the invention are likely to be much cheaper than the alternative Galleri test, and others on the market which are less sensitive. Importantly, the invention can be used to accurately diagnose cancer at an early stage (see FIG. 9); other tests struggle to detect early-stage cancer, as their sensitivity goes up with the cancer stage, whereas the invention maintains exceptionally high sensitivity, specificity, precision and F1 score across all cancer stages.

The invention will now be described by way of example only, with reference to the accompanying drawings.

### BRIEF DESCRIPTION OF THE FIGURES

FIG. 1 shows a principle component analysis of a data set comprising miRNA expression levels for a large number of subjects with and without cancer diagnoses:

FIG. 2 shows a method according to an embodiment:

FIG. 3 shows ROCs and associated AUCs for a first machine learning model employing a panel of 50 miRNAs for determining cancer and not-cancer;

FIG. 4 shows ROCs and associated AUCs for a second machine learning model employing a panel of 50 miRNAs for identifying the type (localisation) of cancer:

FIG. 5 compares results for cancer/not-cancer diagnosis obtained according to an embodiment (employing a 50 miRNA panel) with those of a prior art Galleri, CancerSEEK and Elypta tests:

FIG. 6 shows a graph illustrating how the addition of each miRNA in the full panel of 50 improves AUC values for cancer/not-cancer diagnosis:

FIG. 7 shows results obtained from a machine learning model that predicts cancer/not-cancer from 3 miRNAs:

FIG. 8 shows results obtained from a machine learning model that predicts cancer type from 3 miRNAs.

FIG. 9 shows results obtained from a machine learning model that predicts cancer/not-cancer from 50 miRNAs.

### DETAILED DESCRIPTION

A number of studies have shown that circulating miRNA are useful in the diagnosis of specific cancers. Several datasets across several different cancer types, collected using the same platform (3D-Gene Human miRNA), were available. These different source datasets were combined into an integrated dataset for this work. The source datasets are listed in the references appended to this description. Since each of these datasets were collected using the same platform, their results will be sufficiently consistent that different datasets may be combined. The integrated dataset comprises approximately 20000 samples. In general, a smaller dataset may be used in some embodiments, for example with at least 5000, 10000 or more samples can be used.

Data was median scaled and concatenated before batch correction using the “combat” function from the R package sva. However, no significant difference was noted after this processing and so non-normalised data was used in favour of simplicity. Since all data was collected on the same platform using the same approach in the example, expression levels were directly comparable (but in other embodiments, normalisation and/or batch correction may be useful).

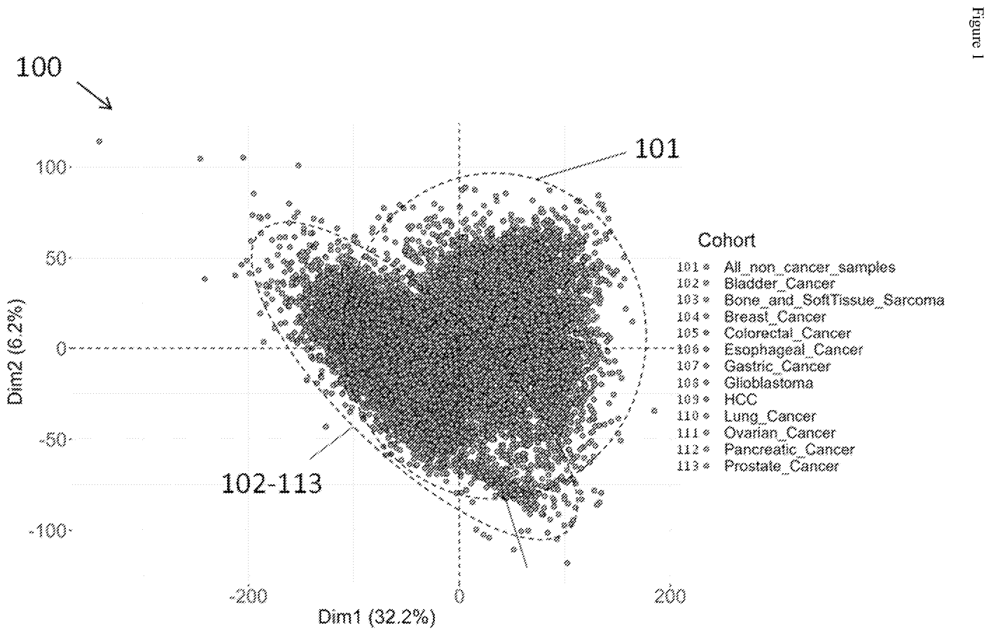

As an initial investigation, a principle components analysis was carried out. FIG. 1 shows the results of a principle component analysis performed on the integrated dataset. Each point corresponds with a set of miRNA expression levels for a sample in the integrated dataset, projected along the first two principle components. FIG. 1 comprises cohorts corresponding with: 101 non cancer samples, 102 bladder cancer, 103 bone and soft tissue sarcoma, 104 breast cancer, 105 colorectal cancer, 106 esophageal cancer, 107 gastric cancer, 108 glioblastoma, 109 hepatocellular carcinoma, 110 lung cancer, 111 ovarian cancer, 112 pancreatic cancer and 113 prostate cancer. It is clear from FIG. 1 that there is, at least, a strong separation in principle component space between the cancer samples 102-113 and non-cancer samples 101 (as illustrated by the ellipses drawn on FIG. 1). In order to develop a diagnostic classification from the miRNA expression levels, a machine learning model was developed.

A panel of 50 miRNA expression levels (listed in Table 1) was selected from the integrated dataset based on importance.

The panel members in Table 1 are selected to both provide good performance in a cancer/no-cancer test (for all cancer stages), and to additionally enable classification of a site of origin of the cancer.

The applicant has found that a panel of 50 miRNAs provides sufficient information for reliable diagnosis of cancer, and of what type of cancer is present. Panels with fewer miRNAs selected from the set of 50 listed herein are also useful, and can provide a reliable classification of samples as indicating cancer or not indicating cancer.

In order to train the machine learning model the integrated dataset was split (randomly) into training, test and validation cohorts (subject to maintaining a distribution across cancer types and stages). The ratio of samples in the training test and validation dataset was approximately 7:2:1, and the total number of samples was ˜20000.

In embodiments a first machine learning model may be used to classify the sample as “cancer” or “no-cancer” and a second machine learning model may be employed to perform further classification on the “cancer” classified samples to identify the type of cancer. In the examples herein, the first machine learning model (i.e. used for determining cancer/not-cancer) is based on a support vector machine with a radial kernel (but other machine learning models may also be used). The first machine learning model in the examples described herein was trained using 10-fold cross validation, repeated 5 times, with sigma and C tuned in a grid.

The support vector machine was trained using labelled data (in which the true classification as “cancer” or “no cancer” is known) to find the best hyperplane that splits the data. Although this is done in a multi-dimensional space, it is easiest to explain using two dimensions (with reference to a Cartesian XY plane).

In this simplified example, on the X axis there is an expression level of one of the miRNA targets from the panel and on the Y axis there is an expression level of a different miRNA target from the panel. If all of the data (using these two miRNAs) are plotted onto the graph, if they are predictive, it will be possible to draw a line to essentially split the data in half. This would mean anything on one side of the line would be cancer anything on the opposite would be no cancer. The model can be tuned to separate the data better—to maximise a distance from the line to the nearest point on either side, for example (which may be referred to as maximum-margin).

In higher dimensional space, the line may be replaced with a hyperplane, so that the approach is generalised to any number of dimensions of input data. It is unlikely that data will perfectly separate and so we can allow certain misclassifications so that the hyperplane ignores certain points (support vectors). This relaxing of the model in turn means it is more likely to fit new data as it is less likely to over fit on the training data. This process is essentially repeated on all combinations of the miRNAs in the panel to build model to classify subjects as having cancer or not having cancer.

In the examples described herein, a feed forward artificial neural network was used for the second machine learning model (i.e. used for classification of cancer type). Rather than separating data based off a hyper plane (separating cancer from non-cancer) the second machine learning model learns through an iterative approach, modifying the weight of elements comprising the model in certain conditions. The feedforward neural network was trained with back propagation using labelled training data (i.e. data for which the type of cancer was known). In the feedforward network there are a number of input nodes equal to the number of miRNAs in the panel, one for each. These then feed into one or more layers of “hidden” nodes. In a simple linear neural network with a single hidden layer, each hidden node receives one or more values from input nodes (e.g. all of them) and determines a weighted sum from the values of the input nodes (e.g. with a linear weight per input to each hidden node). The output from the first layer of hidden nodes may be fed to output nodes (one for each classification to be determined by the model, so twelve in this example). Each output node determines a weighted sum of values of the hidden nodes to determine the classification.

The weights of the nodes in the hidden layer and the output layer are determined by training the neural network. For example data from a first subject is passed through, and back propagation of errors in the classification will cause the weights to be changed so that the classification is correct (or at least, more correct). With each sample in the labelled data set, the weights can be refined until the model is able to predict the sample type on known data. As this is happening, we have a number of tuning parameters which can improve how the training operates. For instance, we can create a penalty to any weights which get heavily reduced, or can make more weights likely to be lower etc. these tuning parameters allow us to fit to the data better, but not over fit so that the model can accurately predict unknown data.

The second machine learning model described in the examples herein was implemented and trained using the H2O framework, but any suitable framework/API may be used.

The example second machine learning model comprised one hidden layer with 1500 nodes. The model was trained with a rectifier activation function which included a dropout ratio of 0.05. Classes were balanced through oversampling and 11 and 12 regularisation of 1×10⁻¹⁰ and 1×10⁻⁹ was used, respectively.

The first and second example machine learning models were trained using data from 14,614 cancer and non-cancer patients (healthy and other diseases), tested on 4,195 patients and further validated in an additional 2058. To assess the performance of the models, the training, test and validation data were kept separate and only used for testing the performance of the machine learning models.

Any other suitable machine learning approach may alternatively be employed for either the first or second machine learning model.

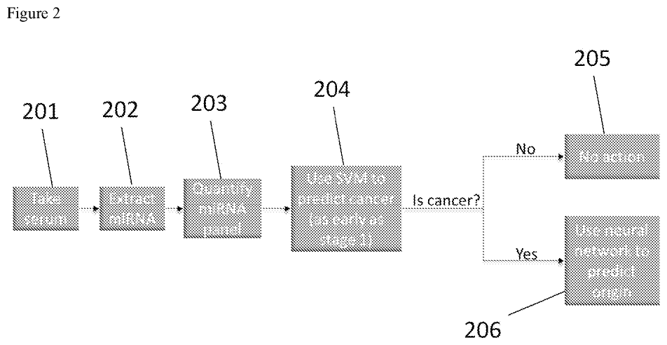

Referring to FIG. 2, steps of an example method according to an embodiment are shown, comprising:
201 Obtaining a biological sample (e.g. serum) from a patient. This step may comprise, for example, receiving a sample that has previously been obtained from a patient, and need not comprise any steps performed on the human or animal body)
202 Extracting miRNA from the biological sample, for example using a magnetic beads;
203 Quantifying miRNA expression levels for each of a plurality of miRNAs comprising a diagnostic panel. This may comprise performing a microarray assay or qRT-PCR analysis;
204 Providing the miRNA expression levels of the panel of miRNAs as an input to a first machine learning model (e.g. a support vector machine). The first machine learning model provides an output indicating whether the panel indicates cancer “yes” or does not indicate cancer “no”.
205 In the event than the first machine learning model outputs a “no”, no further diagnostic or therapeutic action is taken.
206 In the event the first machine learning model outputs a “yes” a second machine learning model is provided with the panel of miRNA expression levels, and the second machine learning model determines a classification indicating what type of cancer is present.

In other embodiments, a single machine learning model can be used to integrate steps 204 and 206, to classify a sample as either indicating “no cancer” or to indicate one of several categories of cancer.

To measure expression levels of the selected miRNAs, the miRNA may be extracted using magnetic beads, and a DNA digestion step performed. Addition of a poly (A) tail to the 3′ end and an adapter is ligated to the 5′ end of the extracted miRNA is performed.

This is then reverse transcribed and the miRNA's abundance measured by i) performing miRNA specific microarray analysis with outputted intensities used for input into the machine learning model; or ii) amplified using universal primers and measured using qPCR with the outputted delta CT values used as an input to the machine learning model. Other techniques may also be used to measure expression levels of the selected miRNAs, which will be apparent to the skilled person and which are outlined above.

The example first machine learning model discussed above (based on a SVM with radial kernel, employing the full 50 miRNA panel) demonstrates 99% specificity and 99% sensitivity for determining cancer or non-cancer across 12 cancers and all stages (0-IV). The machine learning model has a positive predictive value (PPV) of 98.27% and 99.47% in the test and validation cohorts, respectively. Significantly, the second machine learning model employing the same panel of 50 miRNAs is capable of predicting the tumour site of origin with an average AUC (Hand and Till method) of 0.989 and 0.987 and an accuracy of 86.24% and 88.68% in the test and validation cohorts respectively. This corresponds to a macro averaged precision and F1 score of 81.31% and 82.86% in the test cohort, respectively, and a precision and F1 score of 84.72% and 85.68% in the validation cohort, respectively. Interestingly, when considering the top two predicted sites, that is, the two sites predicted as the most likely tumour sites of origin, this accuracy increases to 96.89%. This corresponds to a macro averaged precision and F1 score of 94.17% and 95.21% in the test cohort, respectively, and a precision and F1 score of 95.32% and 95.59% in the validation cohort, respectively.

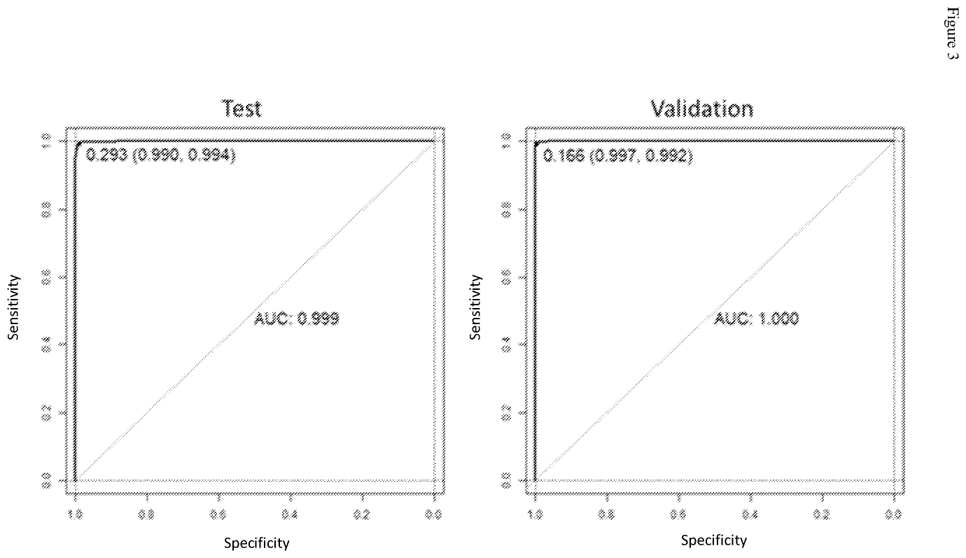

FIG. 3 shows receiver operating characteristic (ROC) curves for the first machine learning model applied to the test data and the validation data (using the full panel of 50 miRNAs as outlined in table 1). The area under the ROC curve (AUC) for the test data for the example first machine learning model is 0.999, and for the validation data is 1.000. For each graph, the inset numbers are cutoff (sensitivity, specificity), with the test cohort having a cutoff probability of 0.293 achieving highest specificity (0.990) and sensitivity (0.994) and the validation cohort having a cutoff probability of 0.166 achieving highest specificity (0.997) and sensitivity (0.992).

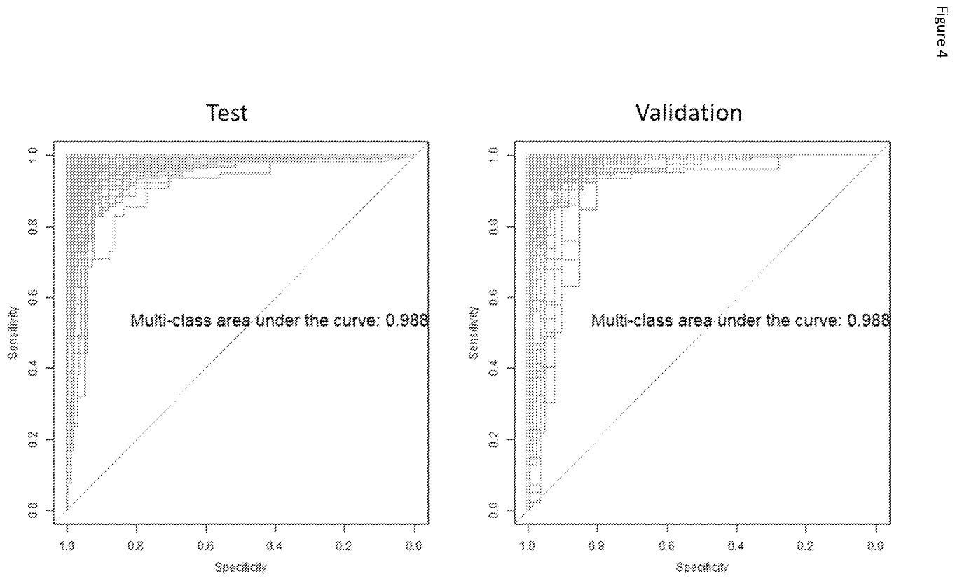

FIG. 4 shows ROC curves for the example second machine learning model discussed above (feedforward network, employing the full 50 miRNA panel), each curve corresponding with a classification category/cancer type (bladder cancer, bone and soft tissue sarcoma, breast cancer, colorectal cancer, esophageal cancer, gastric cancer, glioblastoma, HCC, lung cancer, ovarian cancer, pancreatic cancer, and prostate cancer). The multi-class AUC for both test and validation data was 0.988, and the classification of cancer type had an 88% accuracy.

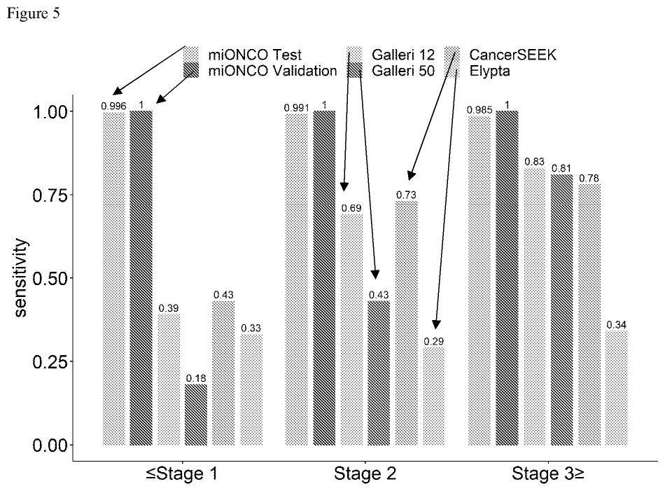

FIG. 5 compares the sensitivity of the example first machine learning model (MiONCO) with the existing Galleri, CancerSEEK and Elypta tests Galleri 12 relates to Galleri performance on 12 cancer types and Galleri 50 being Galleri performance on 50 cancer types. CancerSEEK refers to a test in which the individual sensitivities of each cancer (i.e. colon cancer vs no colon cancer, lung cancer vs no lung cancer) are taken and the median sensitivity plotted (Cohen et al., Science, 2018, 359:6378, p926-930). Elypta uses glycosaminoglycan profiles in urine and/or plasma samples to detect cancer (Gatto et al., Abstract #333361: Detection of any-stage cancer using plasma and urine glycosaminoglycans).

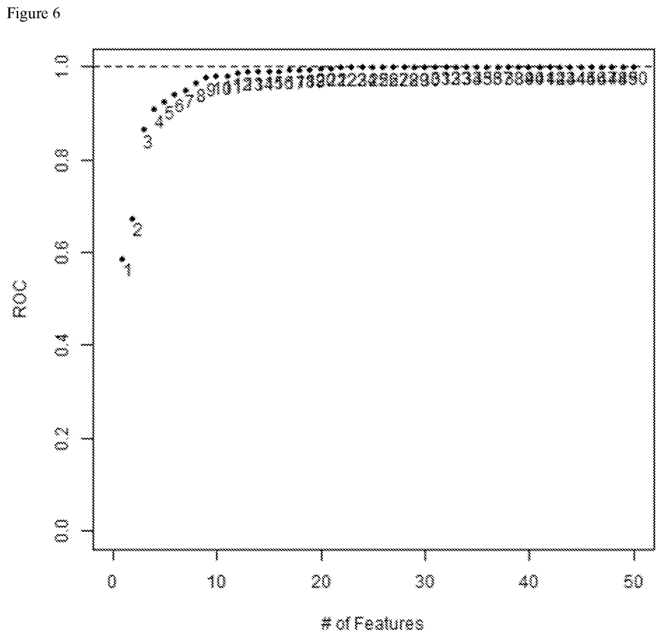

FIG. 6 plots AUC values for different machine learning models that employ different numbers of miRNAs for classification of samples as “cancer” and “no-cancer”. The miRNA panel members listed in Table 1 are set out in order of approximate significance for performing a prediction of cancer/not-cancer. Machine learning models were implemented (and trained) for each subset of the 50 miRNA panel members in accordance with the description of the first machine learning model above, and the results shown are from the training cohort. On the plot, “1” indicates results from a machine learning model trained to identify cancer/not-cancer using the first listed miRNA panel member only, and “2” indicates a machine learning model trained to identify cancer using the first two listed miRNA panel members only, and so on. Once 3 or more miRNA panel members are employed, the AUC is ˜0.85, which is indicative of a very useful diagnostic test. Once more than around 10 panel members are used, the AUC is very high, and there are diminishing returns from the inclusion of further panel members. In some embodiments, different combinations of the 50 panel members may be used. For example, a panel comprising n miRNAs may be selected from any of the 50 listed miRNAs. It is preferable that such a panel comprises the lowest numbered miRNAs, but not essential. For example, a panel comprising n miRNAs may be selected from the first n+i miRNAs, where i is an integer between 1 and 20 (e.g. i=5).

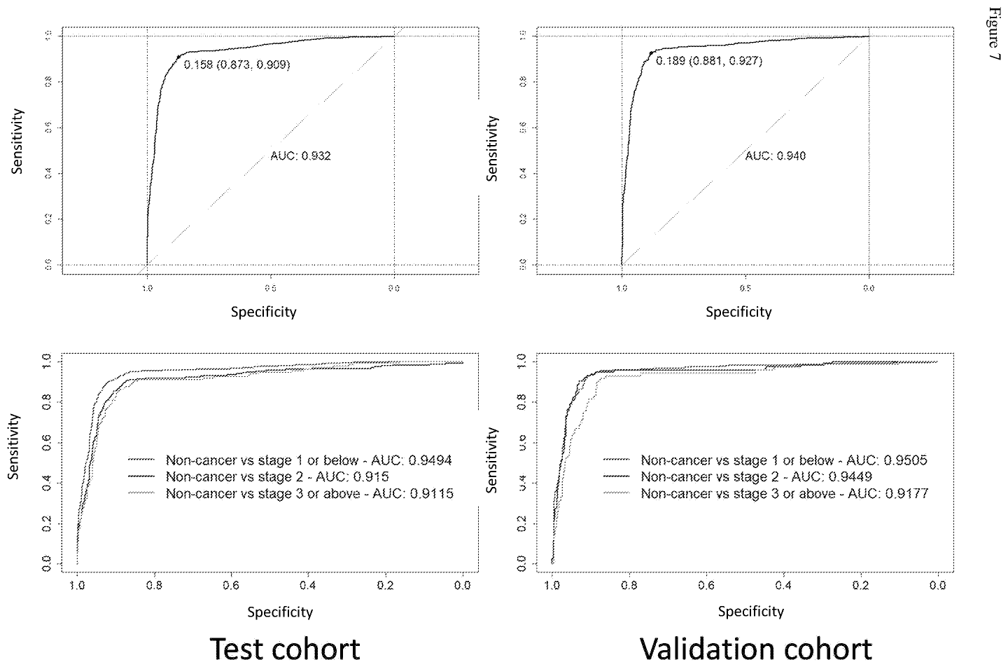

FIG. 7 shows the predictive power of a machine learning model for prediction of cancer (i.e. “cancer” or “no-cancer”) that uses 3 miRNAs selected from the panel (in this instance, the first three panel members, but other panels of 3 may provide similar results). Results are shown for the test cohort and the validation cohort. The AUC for the test 3 miRNA machine learning model (implemented using a SVM with radial kernel, as discussed above) is 0.932 for the test cohort and 0.940 for the validation cohort. FIG. 7 also breaks out the results into different cancer stages. For stage 1 cancer the AUC is 0.949 for the test cohort and 0.951 for the validation cohort, for stage 2 cancer the AUC is 0.915 for the test cohort and 0.945 for the validation cohort, and for stage 3 cancer (and above) the AUC is 0.912 for the test cohort and 0.918 for the validation cohort. Machine learning models according to embodiments therefore provide a reliable diagnostic test for all stages of cancer with 3 miRNAs selected from the panel. Other machine learning approaches (e.g. feedforward neural networks etc) may be used to obtain similar results from the miRNAs identified in the panel.

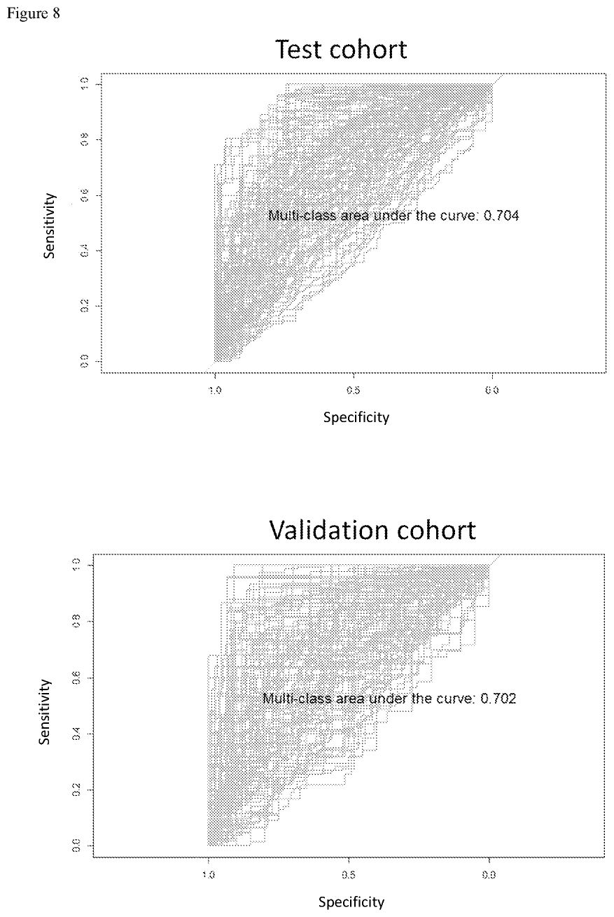

FIG. 8 shows results obtained from a machine learning model for classification of cancer type, trained in accordance with the description of the second machine learning model above, employing 3 miRNAs selected from the panel of Table 1 (in this case, the first three miRNAs). The multi-class AUC for the test data was 0.704 and for the validation data was 0.702. These results show that the 3 panel is useful for prediction of cancer type, if not as accurate as the full panel of 50 miRNAs. It can be expected that similar results can be obtained from other 3 panels selected from the 50 miRNAs listed in Table 1, for example selected from the first 5 or the first 10 miRNAs.

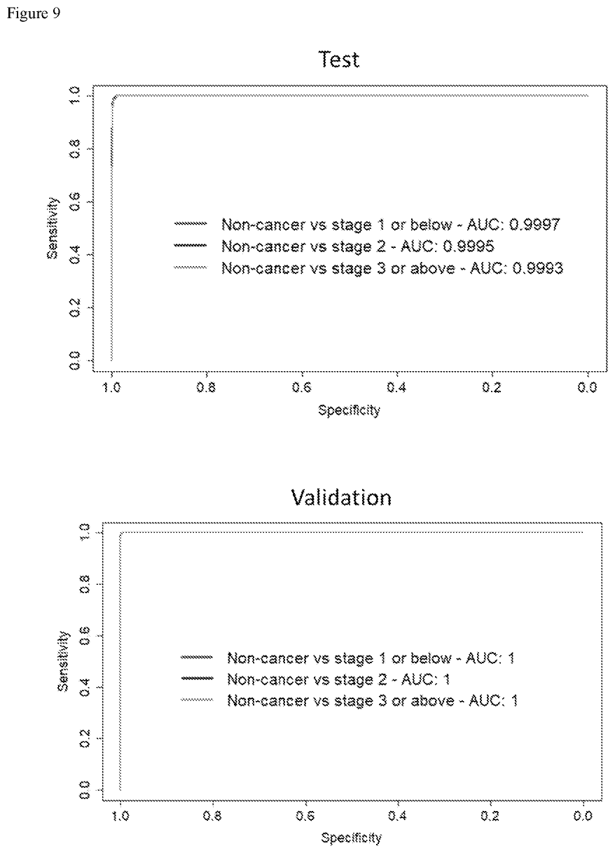

FIG. 9 shows the predictive power of a machine learning model for prediction of cancer (i.e. “cancer” or “no-cancer”) that uses the 50 miRNAs of Table 1. Results are shown for the test cohort and the validation cohort. FIG. 9 also breaks out the results into different cancer stages. For stage 1 cancer the AUC is 0.9997 for the test cohort and 1 for the validation cohort, for stage 2 cancer the AUC is 0.9995 for the test cohort and 1 for the validation cohort, and for stage 3 cancer (and above) the AUC is 0.9993 for the test cohort and 1 for the validation cohort. Machine learning models according to embodiments therefore provide a very reliable diagnostic test for all stages of cancer with the 50 miRNAs of Table 1. Other machine learning approaches (e.g. feedforward neural networks etc) may be used to obtain similar results from the miRNAs identified in the panel.

TABLE 3
class scores for the test cohort referred to herein,
when using the panel of 50 miRNAs of the invention
to determine whether a subject has cancer.

| | Cancer vs Nocancer |
|---|---|
| Sensitivity | 0.993510707 |
| Specificity | 0.989826677 |
| Pos Pred Value | 0.98267009 |
| Neg Pred Value | 0.996207812 |
| Precision | 0.98267009 |
| Recall | 0.993510707 |
| F1 | 0.988060665 |
| Prevalence | 0.367342074 |
| Detection Rate | 0.364958284 |
| Detection | 0.371394517 |
| Prevalence | |
| Balanced | 0.991668692 |
| Accuracy | |

TABLE 4
class scores for the validation cohort referred to
herein, when using the panel of 50 miRNAs of the invention
to determine whether a subject has cancer.

| | Cancer vs |
|---|---|
| | Nocancer |
| Sensitivity | 0.992011 |
| Specificity | 0.99694 |
| Pos Pred Value | 0.99466 |
| Neg Pred Value | 0.995416 |
| Precision | 0.99466 |
| Recall | 0.992011 |
| F1 | 0.993333 |
| Prevalence | 0.364917 |
| Detection Rate | 0.362002 |
| Detection | 0.363946 |
| Prevalence | |
| Balanced | 0.994475 |
| Accuracy | |

TABLE 5
confusion matrix for the test cohort referred to herein,
which shows the amount of subjects predicted to have cancer
or not to have cancer. X-axis labels denote the known outcomes,
whilst y-axis labels denote predicted outcomes.

| | Cancer | Non_Cancer |
|---|---|---|
| Cancer | 1531 | 27 |
| Non_Cancer | 10 | 2627 |

TABLE 6
confusion matrix for the validation cohort referred to herein,
which shows the amount of subjects predicted to have cancer
or not to have cancer. X-axis labels denote the known outcomes,
whilst y-axis labels denote predicted outcomes.

| | Cancer | Non_Cancer |
|---|---|---|
| Cancer | 745 | 4 |
| Non_Cancer | 6 | 1303 |

TABLE 7
class scores for the test cohort referred to herein, when using the panel of 50 miRNAs of the invention to determine a cancer site of origin.

| | | | Pos | Neg | | | | | | | |
|---|---|---|---|---|---|---|---|---|---|---|---|
| | | | Pred | Pred | | | | | Detection | Detection | Balanced |
| | Sensitivity | Specificity | Value | Value | Precision | Recall | F1 | Prevalence | Rate | Prevalence | Accuracy |
| Bladder | 0.932584 | 0.991047 | 0.864583 | 0.995848 | 0.864583 | 0.932584 | 0.897297 | 0.057755 | 0.053861 | 0.062297 | 0.961816 |
| Bone_and_Soft | 0.911111 | 0.991979 | 0.773585 | 0.997312 | 0.773585 | 0.911111 | 0.836735 | 0.029202 | 0.026606 | 0.034393 | 0.951545 |
| Tissue_Sarcoma | | | | | | | | | | | |
| Breast | 0.935484 | 0.998675 | 0.935484 | 0.998675 | 0.935484 | 0.935484 | 0.935484 | 0.020117 | 0.018819 | 0.020117 | 0.96708 |
| Colorectal | 0.705882 | 0.966443 | 0.418605 | 0.989691 | 0.418605 | 0.705882 | 0.525547 | 0.033095 | 0.023361 | 0.055808 | 0.836163 |
| Esophageal | 0.88125 | 0.986966 | 0.886792 | 0.986252 | 0.886792 | 0.88125 | 0.884013 | 0.103829 | 0.091499 | 0.10318 | 0.934108 |
| Gastric | 0.917178 | 0.98107 | 0.928571 | 0.977851 | 0.928571 | 0.917178 | 0.92284 | 0.211551 | 0.19403 | 0.208955 | 0.949124 |
| Glioblastoma | 0.943396 | 0.99664 | 0.909091 | 0.997981 | 0.909091 | 0.943396 | 0.925926 | 0.034393 | 0.032446 | 0.035691 | 0.970018 |
| HCC | 0.822917 | 0.984775 | 0.782178 | 0.988194 | 0.782178 | 0.822917 | 0.80203 | 0.062297 | 0.051265 | 0.065542 | 0.903846 |
| Lung | 0.826531 | 0.985205 | 0.950147 | 0.943333 | 0.950147 | 0.826531 | 0.884038 | 0.25438 | 0.210253 | 0.221285 | 0.905868 |
| Ovarian | 0.825581 | 0.986942 | 0.788889 | 0.989662 | 0.788889 | 0.825581 | 0.806818 | 0.055808 | 0.046074 | 0.058404 | 0.906261 |
| Pancreatic | 0.658537 | 0.988 | 0.6 | 0.990642 | 0.6 | 0.658537 | 0.627907 | 0.026606 | 0.017521 | 0.029202 | 0.823268 |
| Prostate | 0.871345 | 0.990511 | 0.919753 | 0.984046 | 0.919753 | 0.871345 | 0.894895 | 0.110967 | 0.09669 | 0.105127 | 0.930928 |

TABLE 8
class scores for the validation cohort referred to herein, when using the panel of 50 miRNAs of the invention to determine a cancer site of origin.

| | | | Pos | Neg | | | | | | | |
|---|---|---|---|---|---|---|---|---|---|---|---|
| | | | Pred | Pred | | | | | Detection | Detection | Balanced |
| | Sensitivity | Specificity | Value | Value | Precision | Recall | F1 | Prevalence | Rate | Prevalence | Accuracy |
| Bladder | 0.93233 | 0.99435 | 0.99091 | 0.995757 | 0.99091 | 0.93233 | 0.91954 | 0.057257 | 0.053262 | 0.058589 | 0.962291 |
| Bone_and Soft | 0.99091 | 0.995885 | 0.869565 | 0.997253 | 0.869565 | 0.99091 | 0.888889 | 0.029294 | 0.026631 | 0.03626 | 0.952488 |
| Tissue_Sarcoma | | | | | | | | | | | |
| Breast | 0.933333 | 0.998641 | 0.933333 | 0.998641 | 0.933333 | 0.933333 | 0.933333 | 0.019973 | 0.018642 | 0.019973 | 0.965987 |
| Colorectal | 0.84 | 0.965565 | 0.456522 | 0.994326 | 0.456522 | 0.84 | 0.591549 | 0.033289 | 0.027963 | 0.061252 | 0.92782 |
| Esophageal | 0.897436 | 0.994056 | 0.945946 | 0.988183 | 0.945946 | 0.897436 | 0.921053 | 0.13862 | 0.093209 | 0.098535 | 0.945746 |
| Gastric | 0.89375 | 0.984772 | 0.94789 | 0.971619 | 0.94789 | 0.89375 | 0.916667 | 0.213049 | 0.19413 | 0.22397 | 0.939261 |
| Glioblastoma | 1 | 0.995868 | 0.892857 | 1 | 0.892857 | 1 | 0.943396 | 0.033289 | 0.033289 | 0.037284 | 0.997934 |
| HCC | 0.934783 | 0.98156 | 0.767857 | 0.995683 | 0.767857 | 0.934783 | 0.843137 | 0.061252 | 0.057257 | 0.074567 | 0.958171 |
| Lung | 0.854922 | 0.982079 | 0.942857 | 0.951389 | 0.942857 | 0.854922 | 0.896739 | 0.256991 | 0.219707 | 0.233023 | 0.918501 |
| Ovarian | 0.853659 | 0.994366 | 0.897436 | 0.991573 | 0.897436 | 0.853659 | 0.875 | 0.054594 | 0.046605 | 0.051931 | 0.924012 |
| Pancreatic | 0.55 | 0.991792 | 0.647059 | 0.987738 | 0.647059 | 0.55 | 0.594595 | 0.026631 | 0.014647 | 0.022636 | 0.77896 |
| Prostate | 0.951807 | 0.995509 | 0.963415 | 0.994021 | 0.963415 | 0.951807 | 0.957576 | 0.11519 | 0.15193 | 0.19188 | 0.973658 |

TABLE 9
confusion matrix for the test cohort referred to herein, which shows the number of subjects predicted to have cancer
of a certain site of origin. X-axis labels denote the known outcomes, whilst y-axis labels denote predicted outcomes.

| | | Bone_and_SoftTis- | | Colo- | | | Glioblas- | | | | | |
|---|---|---|---|---|---|---|---|---|---|---|---|---|
| | Bladder | sue_Sarcoma | Breast | rectal | Esophageal | Gastric | toma | HCC | Lung | Ovarian | Pancreatic | Prostate |
| Bladder | 86 | 0 | 0 | 0 | 0 | 2 | 1 | 0 | 0 | 1 | 0 | 12 |
| Bone_and_Soft | 0 | 41 | 2 | 1 | 0 | 0 | 0 | 2 | 2 | 1 | 1 | 0 |
| Tissue_Sarcoma | | | | | | | | | | | | |
| Breast | 0 | 1 | 29 | 1 | 0 | 0 | 0 | 0 | 0 | 0 | 0 | 0 |
| Colorectal | 0 | 2 | 0 | 36 | 2 | 5 | 0 | 3 | 17 | 2 | 1 | 0 |
| Esophageal | 0 | 0 | 0 | 1 | 138 | 0 | 0 | 1 | 2 | 6 | 2 | 0 |
| Gastric | 0 | 0 | 0 | 3 | 0 | 293 | 0 | 3 | 17 | 0 | 0 | 0 |
| Glioblastoma | 0 | 0 | 0 | 1 | 0 | 1 | 49 | 0 | 5 | 0 | 0 | 0 |
| HCC | 0 | 0 | 0 | 1 | 2 | 8 | 0 | 76 | 2 | 0 | 6 | 2 |
| Lung | 0 | 0 | 0 | 5 | 2 | 13 | 1 | 0 | 344 | 0 | 0 | 0 |
| Ovarian_{—} | 0 | 0 | 0 | 2 | 7 | 0 | 1 | 2 | 0 | 71 | 0 | 5 |
| Pancreatic | 0 | 1 | 0 | 0 | 9 | 2 | 1 | 9 | 1 | 1 | 31 | 0 |
| Prostate | 3 | 0 | 0 | 0 | 0 | 2 | 0 | 0 | 2 | 4 | 0 | 152 |

TABLE 10
confusion matrix for the test cohort referred to herein, which shows the number of subjects predicted to have cancer
of a certain site of origin. X-axis labels denote the known outcomes, whilst y-axis labels denote predicted outcomes.

| | | Bone_and_SoftTis- | | Colo- | | | Glioblas- | | | | | |
|---|---|---|---|---|---|---|---|---|---|---|---|---|
| | Bladder | sue_Sarcoma | Breast | rectal | Esophageal | Gastric | toma | HCC | Lung | Ovarian | Pancreatic | Prostate |
| Bladder | 40 | 0 | 0 | 0 | 0 | 2 | 1 | 0 | 1 | 0 | 0 | 4 |
| Bone_and_Soft | 0 | 20 | 0 | 2 | 0 | 0 | 0 | 0 | 0 | 1 | 0 | 0 |
| Tissue_Sarcoma | | | | | | | | | | | | |
| Breast | 0 | 1 | 14 | 0 | 0 | 0 | 0 | 0 | 0 | 0 | 0 | 0 |
| Colorectal | 0 | 0 | 1 | 21 | 0 | 2 | 0 | 1 | 11 | 1 | 1 | 0 |
| Esophageal | 0 | 0 | 0 | 0 | 70 | 0 | 0 | 0 | 0 | 2 | 3 | 0 |
| Gastric | 0 | 0 | 0 | 0 | 0 | 137 | 0 | 0 | 6 | 0 | 0 | 0 |
| Glioblastoma | 0 | 0 | 0 | 0 | 1 | 0 | 24 | 0 | 1 | 1 | 0 | 0 |
| HCC | 0 | 0 | 0 | 0 | 0 | 10 | 0 | 43 | 2 | 0 | 4 | 0 |
| Lung | 0 | 1 | 0 | 1 | 2 | 9 | 0 | 0 | 171 | 0 | 1 | 0 |
| Ovarian_{—} | 0 | 0 | 0 | 0 | 3 | 0 | 0 | 0 | 0 | 33 | 0 | 1 |
| Pancreatic | 0 | 0 | 0 | 1 | 2 | 0 | 0 | 2 | 1 | 1 | 11 | 0 |
| Prostate | 3 | 0 | 0 | 0 | 0 | 0 | 0 | 0 | 0 | 2 | 0 | 78 |

Although examples embodiments have been described above, the invention is not limited by those examples, and the scope of protection should be determined with reference to the appended claims.

### SOURCE DATASETS

GSE124158: Asano N, Matsuzaki J, Ichikawa M, Kawauchi J et al. A serum microRNA classifier for the diagnosis of sarcomas of various histological subtypes. Nat Commun 2019 Mar. 21; 10 (1): 1299. PMID: 30898996
GSE137140: Asakura K, Kadota T, Matsuzaki J, Yoshida Y et al. A miRNA-based diagnostic model predicts resectable lung cancer in humans with high accuracy. Commun Biol 2020 Mar. 19; 3 (1): 134. PMID: 32193503
GSE122497: Sudo K, Kato K, Matsuzaki J, Boku N et al. Development and Validation of an Esophageal Squamous Cell Carcinoma Detection Model by Large-Scale MicroRNA Profiling. JAMA Netw Open 2019 May 3; 2 (5): e194573. PMID: 31125107
GSE112264: Urabe F, Matsuzaki J, Yamamoto Y, Kimura T et al. Large-scale Circulating microRNA Profiling for the Liquid Biopsy of Prostate Cancer. Clin Cancer Res 2019 May 15; 25 (10): 3016-3025. PMID: 30808771
GSE113486: Usuba W, Urabe F, Yamamoto Y, Matsuzaki J et al. Circulating miRNA panels for specific and early detection in bladder cancer. Cancer Sci 2019 Jan; 110 (1): 408-419. PMID: 30382619
GSE113740: Yamamoto Y, Kondo S, Matsuzaki J, Esaki M et al. Highly Sensitive Circulating MicroRNA Panel for Accurate Detection of Hepatocellular Carcinoma in Patients With Liver Disease. Hepatol Commun 2020 Feb; 4 (2): 284-297. PMID: 32025611
GSE139031: Ohno M, Matsuzaki J, Kawauchi J, Aoki Y et al. Assessment of the Diagnostic Utility of Serum MicroRNA Classification in Patients With Diffuse Glioma. JAMA Netw Open 2019 Dec. 2; 2 (12): e1916953. PMID: 31808923
GSE164174: Abe S, Matsuzaki J, Sudo K, Oda I et al. A novel combination of serum microRNAs for the detection of early gastric cancer. Gastric Cancer 2021 Jul; 24 (4): 835-843. PMID: 33743111
GSE106817: Yokoi A, Matsuzaki J, Yamamoto Y, Yoneoka Y et al. Integrated extracellular microRNA profiling for ovarian cancer screening. Nat Commun 2018 Oct. 17; 9 (1): 4319. PMID: 30333487

1. A method of diagnosing a cancer in a subject, wherein the method comprises:
i. providing a biological sample obtained from the subject;
ii. determining the expression level of at least three miRNAs in the sample obtained from the subject;
iii. using the results from (ii) to determine if the subject has a cancer.

2. The method of claim 1, wherein the at least three miRNAs are selected from Table 1.

3. The method of claim 1 or 2, wherein the at least three miRNAs comprise or consist of three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 of the miRNAs in Table 1.

4. The method of any of claims 1-3, wherein the at least three miRNAs may comprise or consist of miRNAs having the sequence of SEQ ID NO: 1, SEQ ID NO: 2 and SEQ ID NO: 3.

5. The method of any of claims 1-3, wherein the at least three miRNAs may comprise or consist of miRNAs having the sequences of SEQ ID NO: 1, SEQ ID NO: 3, SEQ ID NO: 4, SEQ ID NO: 5, and SEQ ID NO: 6.

6. The method of any of claims 1-3, wherein the at least three miRNAs may comprise or consist of miRNAs having the sequence of all of SEQ ID NOs: 1-50.

7. The method of any of claims 1-6, wherein a machine learning model is used to determine if the subject has cancer from the expression levels of the at least three miRNAs.

8. The method of claim 7, wherein the machine learning model comprises a support vector machine.

9. The method of claim 8, wherein the vector machine employs a radial kernel.

10. The method of any of claims 7-9, wherein the method comprises using a second machine learning model to identify what type of cancer is present in the event that the first machine learning model determines that the subject has cancer.

11. The method of claim 10, wherein the second machine learning model comprises a feedforward artificial neural network.

12. The method of claim 11, wherein the feedforward artificial neural network comprises a single hidden layer, and/or at least one hidden layer.

13. A method of determining the localisation of a cancer in a subject, comprising:
i. providing a biological sample obtained from the subject;
ii. determining the expression level of a panel of miRNAs, the panel comprising miRNAs having the sequences of at least three of, up to all of, SEQ ID NOs 1-50, in the sample obtained from the subject; and
iii. using the results from (ii) to determine a location of the cancer, optionally wherein a second machine learning model is used to identify the location of the cancer in the event that the first machine learning model determines that the subject has cancer, optionally wherein the second machine learning model comprises a feedforward artificial neural network.

14. The method of claim 13, wherein the at least three miRNAs comprise or consist of miRNAs having the sequence of all miRNAs of Table 2.

15. The method of claim 13, wherein the three or more miRNAs comprise or consist of the 30 miRNAs of Table 2, the first 25 miRNAs of Table 2, the first 20 miRNAs of Table 2, the first 15 miRNAs of Table 2, or the first 10 miRNAs of Table 2.

16. The method of any preceding claim, wherein the biological sample is a biological fluid, such as a blood, plasma, serum, urine or cerebrospinal fluid sample.

17. The method of any preceding claim, wherein the cancer is a solid cancer, optionally wherein the solid cancer is selected from the group consisting of: bladder cancer, bone and soft tissue sarcoma, breast cancer, colorectal cancer, oesophageal cancer, gastric cancer, glioblastoma, hepatocellular carcinoma (HCC), lung cancer, ovarian cancer, pancreatic cancer, or prostate cancer.

18. The method of any preceding claim, wherein the cancer is stage 0 cancer, stage 1 cancer, stage 2 cancer, stage 3 cancer or stage 4 cancer

19. A method of treating a subject with cancer, wherein the method comprises:
i. providing a biological sample obtained from the subject;
ii. determining the expression level of at least three miRNAs in the sample obtained from the subject;
iii. using the results from (ii) to determine if the subject has a cancer; and
iv. administering an anti-cancer therapeutic to the subject if the subject is determined to have cancer.

20. The method of claim 17, wherein the at least three miRNAs may comprise or consist of miRNAs having the sequence of SEQ ID NO: 1, SEQ ID NO: 2 and SEQ ID NO: 3.

21. The method of claim 17, wherein the at least three miRNAs may comprise or consist of miRNAs having the sequences of SEQ ID NO: 1, SEQ ID NO: 3, SEQ ID NO: 4, SEQ ID NO: 5, and SEQ ID NO: 6.

22. The method of claim 17, wherein the at least three miRNAs may comprise or consist of miRNAs having the sequence of all of SEQ ID NOs: 1-50.

23. A kit for carrying out the method of any of claims 1-22, wherein the kit comprises one or more reagents for determining the expression level of the at least three miRNAs.

24. The kit of claim 24, wherein the reagents are one or more oligonucleotide primers specific for each of the at least three miRNAs.

25. the kit of claim 23 or claim 24, further comprising a set of instructions which set out to the reader the details and information required to perform the method of any of claims 1-23.


SEQUENCE LISTING (50 sequences)

SEQ ID NO: 1 - RNA, 23 nt, Homo sapiens
  source 1..23: mol_type = other RNA
ttgctctgctcccccgcccccag

SEQ ID NO: 2 - RNA, 23 nt, Homo sapiens
  source 1..23: mol_type = other RNA
ctgggcccgcggcgggcgtgggg

SEQ ID NO: 3 - RNA, 25 nt, Homo sapiens
  source 1..25: mol_type = other RNA
gtgaggcggggccaggagggtgtgt

SEQ ID NO: 4 - RNA, 17 nt, Homo sapiens
  source 1..17: mol_type = other RNA
gagactggggtggggcc

SEQ ID NO: 5 - RNA, 17 nt, Homo sapiens
  source 1..17: mol_type = other RNA
ctccgggacggctgggc

SEQ ID NO: 6 - RNA, 21 nt, Homo sapiens
  source 1..21: mol_type = other RNA
agggagggacgggggctgtgc

SEQ ID NO: 7 - RNA, 22 nt, Homo sapiens
  source 1..22: mol_type = other RNA
ggggggtgtggagccagggggc

SEQ ID NO: 8 - RNA, 22 nt, Homo sapiens
  source 1..22: mol_type = other RNA
ctcgggagggcatgggccaggc

SEQ ID NO: 9 - RNA, 22 nt, Homo sapiens
  source 1..22: mol_type = other RNA
tgggagggcgtggatgatggtg

SEQ ID NO: 10 - RNA, 19 nt, Homo sapiens
  source 1..19: mol_type = other RNA
cggggccagagcagagagc

SEQ ID NO: 11 - RNA, 22 nt, Homo sapiens
  source 1..22: mol_type = other RNA
ttcagatcccagcggtgcctct

SEQ ID NO: 12 - RNA, 23 nt, Homo sapiens
  source 1..23: mol_type = other RNA
ggaggcgcaggctcggaaaggcg

SEQ ID NO: 13 - RNA, 23 nt, Homo sapiens
  source 1..23: mol_type = other RNA
gccccggcgcgggcgggttctgg

SEQ ID NO: 14 - RNA, 21 nt, Homo sapiens
  source 1..21: mol_type = other RNA
cgggccggaggtcaagggcgt

SEQ ID NO: 15 - RNA, 23 nt, Homo sapiens
  source 1..23: mol_type = other RNA
tgagtggggctcccgggacggcg

SEQ ID NO: 16 - RNA, 17 nt, Homo sapiens
  source 1..17: mol_type = other RNA
ggcgggtgcgggggtgg

SEQ ID NO: 17 - RNA, 18 nt, Homo sapiens
  source 1..18: mol_type = other RNA
ggtgggcttcccggaggg

SEQ ID NO: 18 - RNA, 23 nt, Homo sapiens
  source 1..23: mol_type = other RNA
tgggccagggagcagctggtggg

SEQ ID NO: 19 - RNA, 20 nt, Homo sapiens
  source 1..20: mol_type = other RNA
cgggcgtggtggtgggggtg

SEQ ID NO: 20 - RNA, 21 nt, Homo sapiens
  source 1..21: mol_type = other RNA
tgagggacccaggacaggaga

SEQ ID NO: 21 - RNA, 22 nt, Homo sapiens
  source 1..22: mol_type = other RNA
ggctacaacacaggacccgggc

SEQ ID NO: 22 - RNA, 23 nt, Homo sapiens
  source 1..23: mol_type = other RNA
tgagcaccacacaggccgggcgc

SEQ ID NO: 23 - RNA, 22 nt, Homo sapiens
  source 1..22: mol_type = other RNA
atatacagggggagactctcat

SEQ ID NO: 24 - RNA, 22 nt, Homo sapiens
  source 1..22: mol_type = other RNA
ctcctggggcccgcactctcgc

SEQ ID NO: 25 - RNA, 22 nt, Homo sapiens
  source 1..22: mol_type = other RNA
cgcaggggccgggtgctcaccg

SEQ ID NO: 26 - RNA, 19 nt, Homo sapiens
  source 1..19: mol_type = other RNA
ctggggagtggctggggag

SEQ ID NO: 27 - RNA, 18 nt, Homo sapiens
  source 1..18: mol_type = other RNA
ggtggggggtgttgtttt

SEQ ID NO: 28 - RNA, 22 nt, Homo sapiens
  source 1..22: mol_type = other RNA
actggacttagggtcagaaggc

SEQ ID NO: 29 - RNA, 23 nt, Homo sapiens
  source 1..23: mol_type = other RNA
agctacattgtctgctgggtttc

SEQ ID NO: 30 - RNA, 22 nt, Homo sapiens
  source 1..22: mol_type = other RNA
gctgcgggctgcggtcagggcg

SEQ ID NO: 31 - RNA, 24 nt, Homo sapiens
  source 1..24: mol_type = other RNA
taggtagagtgtgaggaggaggtc

SEQ ID NO: 32 - RNA, 20 nt, Homo sapiens
  source 1..20: mol_type = other RNA
accaggaggctgaggcccct

SEQ ID NO: 33 - RNA, 18 nt, Homo sapiens
  source 1..18: mol_type = other RNA
gggtgagggcaggtggtt

SEQ ID NO: 34 - RNA, 22 nt, Homo sapiens
  source 1..22: mol_type = other RNA
tagcagcacgtaaatattggcg

SEQ ID NO: 35 - RNA, 18 nt, Homo sapiens
  source 1..18: mol_type = other RNA
tgaggcgggggggcgagc

SEQ ID NO: 36 - RNA, 22 nt, Homo sapiens
  source 1..22: mol_type = other RNA
atatacagggggagactcttat

SEQ ID NO: 37 - RNA, 22 nt, Homo sapiens
  source 1..22: mol_type = other RNA
aaggggcagggacgggtggccc

SEQ ID NO: 38 - RNA, 17 nt, Homo sapiens
  source 1..17: mol_type = other RNA
ggatggaggaggggtct

SEQ ID NO: 39 - RNA, 17 nt, Homo sapiens
  source 1..17: mol_type = other RNA
cgcgccgggcccgggtt

SEQ ID NO: 40 - RNA, 21 nt, Homo sapiens
  source 1..21: mol_type = other RNA
tggctgttggagggggcaggc

SEQ ID NO: 41 - RNA, 21 nt, Homo sapiens
  source 1..21: mol_type = other RNA
atgcctcccccggccccgcag

SEQ ID NO: 42 - RNA, 22 nt, Homo sapiens
  source 1..22: mol_type = other RNA
ctgggggtggggggctgggcgt

SEQ ID NO: 43 - RNA, 20 nt, Homo sapiens
  source 1..20: mol_type = other RNA
agagatgaagcgggggggcg

SEQ ID NO: 44 - RNA, 21 nt, Homo sapiens
  source 1..21: mol_type = other RNA
tagcagcacagaaatattggc

SEQ ID NO: 45 - RNA, 23 nt, Homo sapiens
  source 1..23: mol_type = other RNA
tgggaggggagaggcagcaagca

SEQ ID NO: 46 - RNA, 22 nt, Homo sapiens
  source 1..22: mol_type = other RNA
actcggcgtggcgtcggtcgtg

SEQ ID NO: 47 - RNA, 22 nt, Homo sapiens
  source 1..22: mol_type = other RNA
agtgggaggccagggcacggca

SEQ ID NO: 48 - RNA, 20 nt, Homo sapiens
  source 1..20: mol_type = other RNA
gtgagtcagggtggggctgg

SEQ ID NO: 49 - RNA, 21 nt, Homo sapiens
  source 1..21: mol_type = other RNA
ctgggtgagggcatctgtggt

SEQ ID NO: 50 - RNA, 23 nt, Homo sapiens
  source 1..23: mol_type = other RNA
cacacaggaaaagcggggccctg